(19) United States
(12) Patent Application Publication
(10) Pub. No.: US 2014/0046062 A1
(43) Pub. Date: Feb. 13, 2014

(54) PROCESS FOR NUCLEOSIDES

(71) Applicants: Bandi Parthasaradhi Reddy, Hyderabad (IN); Kura Rathnakar Reddy, Hyderabad (IN); Dasari Muralidhara Reddy, Hyderabad (IN); Rapolu Raji Reddy, Hyderabad (IN); Bandi Vamsi Krishna, Hyderabad (IN)

(72) Inventors: Bandi Parthasaradhi Reddy, Hyderabad (IN); Kura Rathnakar Reddy, Hyderabad (IN); Dasari Muralidhara Reddy, Hyderabad (IN); Rapolu Raji Reddy, Hyderabad (IN); Bandi Vamsi Krishna, Hyderabad (IN)

(21) Appl. No.: 13/520,955

(22) PCT Filed: Jan. 8, 2010
(86) PCT No.: PCT/IN10/00014

§ 371 Date: Jul. 6, 2012

(51) Int. Cl. C07D 411/04

(52) U.S. Cl. CPC C07D 411/04
USPC 544/317

(57) ABSTRACT

The present invention relates to improved process for the preparation of lamivudine or emtricitabine. Thus, (1′R,2′S,5′R)-menthyl-5(R,S)-acetoxy-[1,3]-oxathiolane-2(R)-carboxylate is reacted with N-propinoyl cytosine in hexamethyl disilazane and then added trityl perchlorate to obtain a solid containing (1′R,2′S,5′R)-menthyl-5S-(N-4″-propionylcytosin-1″-yl)-[1,3]-oxathiolane-2R-carboxylate. The solid obtained above is reacted with methane sulfonic acid to obtain (2R,5S)-5-(4-amino-2-oxo-2H-pyrimidin-1-yl)-[1,3]-oxathiolane-2-carboxylic acid, 2S-isopropyl-5R-methyl-1R-cyclohexyl ester. The above compound is reduced with sodium borohydride to obtain lamivudine.

### FIELD OF THE INVENTION

The present invention relates to improved process for the preparation of nucleosides.

### BACKGROUND OF THE INVENTION

Antiretroviral activity of 2-substitued-5-substitued-1,3-oxathiolanes were disclosed in U.S. Pat. Nos. 5,047,407 and 5,538,975. Of the compounds, Lamivudine, chemically (2R-cis)-4-amino-1-[2-(hydroxymethyl)-1,3-oxathiolan-5-yl]-2(1H)-pyrimidinone is marketed under the brand EPIVIR and Emitricitabine, chemically 4-amino-5-fluoro-1-[(2R,5S)-2-hydroxymethyl)-1,3-oxathiolan-5yl]-2(1H)-pyrimidionone is marketed under the brand EMTRIVA. Lamivudine is represented by the following structure:

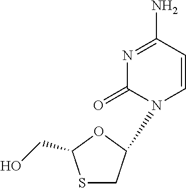 [SMILES: Nc1ccn([C@@H]2CS[C@H](CO)O2)c(=O)n1]

Emitricitabine is represented by the following structure:

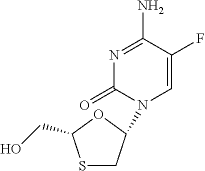 [SMILES: Nc1nc(=O)n([C@@H]2CS[C@H](CO)O2)cc1F]

(2R-cis)-4-Amino-1-[2-(hydroxymethyl)-1,3-oxathiolan-5-yl]-2(1H)-pyrimidinone is also useful in the treatment of hepatitis B infection as disclosed in US RE39155. WO Patent Publication No. 92/20344 disclosed a method of treatment of HIV infection and other viral infection with lamivudine in combination with other antiviral agents such as Zidovudine, chemically 3′-azido-3′-deoxythymidine.

International patent application publication no. WO 2004/064845 disclosed a method of treatment of HIV infection and other viral infection with emtricitabine in combination with other antiviral agents such as tenofovir disoproxil fumarate.

Lamivudine may be prepared using the procedures described in U.S. Pat. No. 5,047,407 (herein after referred as '407 patent). '407 patent disclosed the 1,3-oxathiolane derivatives; their geometric (cis/trans) and optical isomers. The patent described the preparation of 2-substitued-5-substitued-1,3-oxathiolanes. '407 patent described the preparation of individual stereoisomers of 2-substitued-5-substitued-1,3-oxathiolanes from stereoisomerically pure raw materials or intermediates.

U.S. Pat. No. 5,538,975 described the process for the preparation of emtricitabine.

U.S. Pat. No. 5,248,776 described an asymmetric process for the synthesis of enantiomerically pure β-L-(−)-1,3-oxathiolone-nucleosides starting from optically pure 1,6-thioanhydro-L-gulose, which in turn can be easily prepared from L-Gulose. The condensation of the 1,3-oxathiolane derivative with the heterocyclic base is carried out in the presence of a Lewis acid, most preferably SnC14, to give the [2R,5R] and [2R,5S] diastereomers that are then separated chromatographically.

International patent application publication no. WO92/20669 disclosed a diastereoselective process for producing optically active cis-nucleoside analogues and derivatives of formula (I)

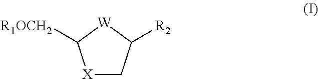

wherein W is S, S═O, SO₂, or O;

### X is S, S═O, SO₂ or O;

R1 is hydrogen or acyl; and
R2 is a desired purine or pyrimidine base or an analogue or derivative thereof; the process comprising the step of reacting the desired purine or pyrimidine base or analogue thereof with an intermediate of formula (IIa) or (IIb)

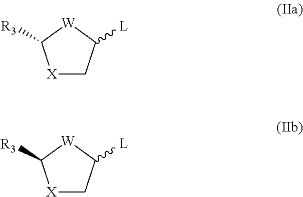

wherein
R3 is a substituted carbonyl or carbonyl derivative; and
L is a leaving group;
using a Lewis acid such as iodotrimethylsilane (TMSI) or trimethylsilyl triflate (TMSOTf).

The process of WO92/20669 allows the stereo-controlled synthesis of a racemic cis-nucleoside analogue from an equimolar mixture of (IIa) and (IIb), and of a given enantiomer of a desired cis-nucleoside analogue in high optical purity if the starting material is optically pure (IIa) or (IIb). However, the WO92/20669 process relies on the use of a Lewis acid of formula (III).

There are a number of disadvantages associated with the use of such Lewis acids. In particular, they are highly reactive and unstable compounds and there are therefore hazards associated with their use. Furthermore, they are expensive and have significant toxic effects. These disadvantages are of particular importance in relation to the large-scale production of nucleoside analogues in factory processes.

International patent application publication no. WO95/29174 disclosed a stereoselective process for producing cis-nucleoside analogues and derivatives of formula (I)

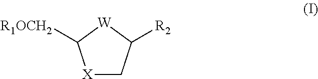

wherein W is S, S═O, SO₂, or O;

### X is S, S═O, SO₂, or O;

R1 is hydrogen or acyl; and
R2 is a purine or pyrimidine base or an analogue thereof;
the process comprising the step of glycosylating the purine or pyrimidine base or analogue or derivative thereof with an intermediate of formula (IVa) or (IVb)

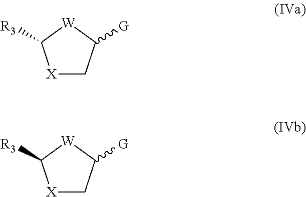

wherein R3 is a substituted carbonyl or carbonyl derivative; and G represents halo, cyano or R⁹SO2—where R⁹ represents alkyl optionally substituted by one or more halo, or optionally substituted phenyl;
characterized in that the glycosylation reaction is effected without the addition of a Lewis acid catalyst.

U.S. Pat. No. 6,600,044 described a method for converting the undesired trans-1,3-oxathiolane nucleoside to the desired cis isomer by a method of anomerization or transglycosylation and the separation of the hydroxyl-protected form of cis-, trans-(−)-nucleosides by fractional crystallization of their hydrochloride, hydrobromide, methanesulfonate salts.

U.S. Pat. No. 6,175,008 described the preparation of lamivudine by reacting mercaptoacetaldehyde dimer with glyoxalate and further with silylated pyrimidine base to give mainly the cis-isomer by using an appropriate Lewis acid, like TMS-I, TMS-Tf, TiCl₄ et cetera. However the stereoselectivity is not absolute and although the cis isomer is obtained in excess, this process still requires its separation from the trans isomer. The separation of the diastereomers is done by acetylation and chromatographic separation followed by deacetylation. Further separation of the enantiomer of the cis-isomer is not mentioned.

WO Patent Publication No. 2008/053496 disclosed a process for the resolution of cis(±)-4-amino-1-[2-(hydroxymethyl)-1,3-oxathiolan-5-yl]-2(1H)-pyrimidinone by using S-1,1′-bi-2-naphthol ((S)-BINOL). We have found that the process is not reproducible.

International co-pending application no. PCT/IN08/000823 disclosed a process for the resolution of cis(±)-4-amino-1-[2-(hydroxymethyl)-1,3-oxathiolan-5-yl]-2(1H)-pyrimidinone by using S(+)-1,1′-binaphthyl-2,2′-diyl hydrogen phosphate ((S)-BNPPA). According to the application, cis(±)-4-Amino-1-[2-(hydroxymethyl)-1,3-oxathiolan-5-yl]-2(1H)-pyrimidinone was reacted with (S)-BNPPA in an alcohol solvent such as methanol, selectively crystallizing 4-amino-1-[(2R,5S)-2-(hydroxymethyl)-[1,3]-oxathiolan-5-yl]-(1H)-pyrimidin-2-one-S-BNPPA complex, treating the complex with an acid or base to obtain lamivudine.

We have discovered a novel process for preparation of nucleosides. The object of the present invention is to provide an improved and commercially viable process for preparation of nucleosides.

### SUMMARY OF THE INVENTION

In accordance the present invention, there is provided a process for the preparation of nucleosides, which comprises:

a) reacting the compound of formula I:

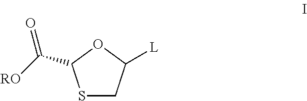

wherein the R is independently hydrogen and optionally substituted alkyl groups and L is leaving group with a compound of formula II:

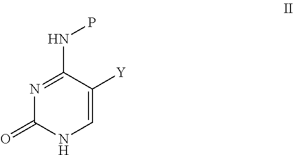

wherein the P is protecting group and Y is hydrogen or fluorine to obtain a compound of formula III:

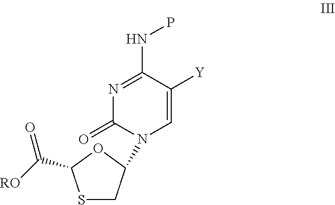

wherein the R, P and Y are defined above,
   b) treating the compound of formula III with an acid to obtain a compound of formula IV;

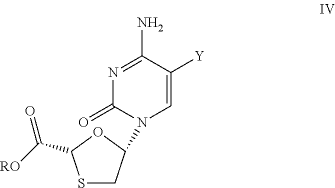

wherein the R and Y are defined above,
   c) reducing the compound of formula IV with a reducing agent to obtain a compound of formula V,

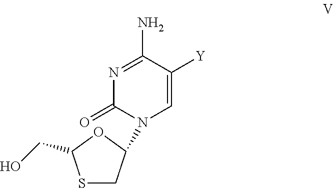

wherein the Y is defined above,
   d) optionally, converting the compound of formula V to a salts thereof.

### DETAILED DESCRIPTION OF THE INVENTION

According to the present invention, there is provided a process for the preparation of nucleosides, which comprises:

a) reacting the compound of formula I:

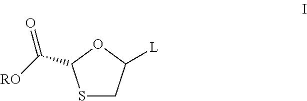

wherein the R is independently hydrogen and optionally substituted alkyl groups and L is leaving group with a compound of formula II:

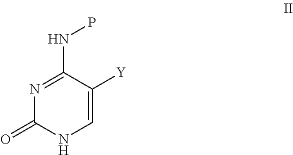

wherein the P is protecting group and Y is hydrogen or fluorine to obtain a compound of formula III:

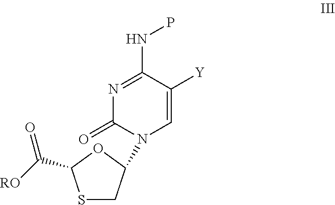

wherein the R, P and Y are defined above,
   b) treating the compound of formula III with an acid to obtain a compound of formula IV;

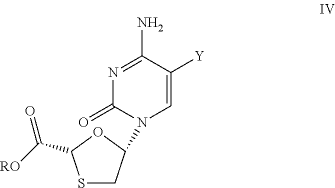

wherein the R and Y are defined above,
   c) reducing the compound of formula IV with a reducing agent to obtain a compound of formula V,

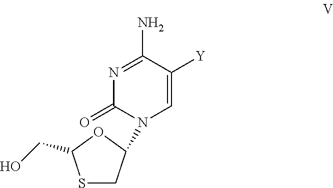

wherein the Y is defined above,
   d) optionally, converting the compound of formula V to a salts thereof.

Preferable R is an optionally substituted alkyl group. The more preferable substituted alkyl group is a chiral auxiliary.

The term “chiral auxiliary” describes an asymmetric molecule that is used to effect the chemical resolution of a racemic mixture. Such chiral auxiliaries may possess one chiral center such as .alpha.-methylbenzylamine or several chiral centers such as menthol. The purpose of the chiral auxiliary, once built-into the starting material, is to allow simple separation of the resulting diastereomeric mixture. See, for example, J Jacques et al., Enantiomers, Racemates and Resolutions, pp. 251-369, John Wiley & Sons, New York (1981).

Preferably the chiral auxiliary is selected from (d)-menthol, (l)-menthol, (d)-8-phenylmenthyl, (l)-8-phenylmenthyl, (+)-norephedrine and (−)-norephedrine. The more preferable chiral auxiliary is (d)-menthol or (l)-menthol.

Leaving group i.e., an atom or a group which is displaceable upon reaction with an appropriate purine or pyrimidine base, with or without the presence of a Lewis acid. Preferable leaving groups are acyloxy groups e.g., acetoxy, alkoxy groups, e.g., alkoxy carbonyl groups such as ethoxy carbonyl; halogens such as iodine, bromine, chlorine, or fluorine. More preferable leaving group is acetoxy.

Preferable protecting groups “P” are propionyl, butanoyl, pentanoyl, hexanoyl, tosyl, mesyl or benzoyl and, more preferable protecting group is propionyl.

The reaction in step (a) may preferably be carried out compound of formula I is reacted with protected cytosine compound of formula II. The protected cytosine compound of formula II is preferably silylated with hexamethyl disilazane in the presence of organic acid such as methane sulfonic acid and aromatic solvent. Preferable aromatic solvent is selected from benzene, toluene or xylene and, more preferable aromatic solvent is toluene.

Preferably, the acid used in step (b) is selected from methane sulfonic acid, ethane sulfonic acid, p-toluene sulfonic acid, acetic acid, formic acid, hydrochloric acid, sulfuric acid or phosphoric acid. More preferable acid is methane sulfonic acid.

The reaction in step (b) may preferably be carried out in a solvent or mixture thereof. Preferable solvent is selected from ether solvents such as diisopropyl ether, di-tert-butyl ether, diethyl ether, 1,4-dioxane, ethyl tert-butyl ether, methyl tert-butyl ether and tetrahydrofuran, and more preferable solvent is diisopropyl ether.

Preferably, the reducing agent used in step (c) is selected from sodium borohydride, lithium aluminium hydride, sodium amalgam, oxalic acid, formic acid or diisobutylalumiminum hydride and more preferable reducing agent is sodium borohydride.

Preferable acid addition salts prepared in step (d) are hydrochloric acid, sulfuric acid, methane sulfonic acid, succinic acid, salicylic acid, malic acid and p-toluene sulfonic acid. More preferable acid addition salt is succinic acid.

The process for the preparation of nucleosides may be represented by the following scheme:

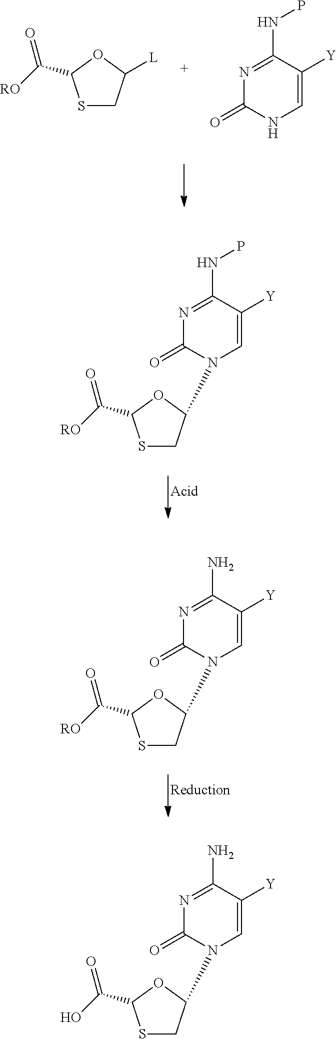

The invention will now be further described by the following examples, which are illustrative rather than limiting.

### EXAMPLES

### Example 1

### Preparation of N-Propionyl Cytosine

Cytosine (150 gm) was added toluene (600 ml) at room temperature and the contents were heated. Distilled off the solvent under atmospheric pressure at 110° C. and the contents were cooled to 75° C. Pyridine (135 gm) and dimethylaminopyridine (2 gm) was added to the reaction mass at 75° C. To the reaction mass was added propionic anhydride (190 gm) with toluene (400 ml) at 75° C. for 1 hour. The reaction mass was cooled to room temperature, filtered. The solid obtained was washed with toluene and further washed with water to obtain wet solid. To the wet solid was suspended in water (3000 ml) and triethylamine (7 gm) was added at room temperature. The reaction mass was stirred for 3 hours at room temperature, the separated solid was filtered and washed with water. The solid was dried at 50 to 55° C. under reduced pressure for 6 hours to obtain 210 gm of N-propionyl cytosine.

### Example 2

### Preparation of (1′R,2′S,5′R)-menthyl-5(R,S)-acetoxy-[1,3]-oxathiolane-2R-carboxylate

(1′R,2′S,5′R)-menthyl-5R-hydroxy-[1,3]-oxathiolane-2(R)-carboxylate (75 gm) was added to diisopropyl ether (300 ml) at room temperature. The contents were cooled to 5° C. and then added pyridine (26 gm) followed by dimethyl amino pyridine (10 mg). A mixture of acetic anhydride (30 gm) and diisopropyl ether (75 ml) was added to the reaction mass under stirring at 5° C. The temperature of the reaction mass was raised to room temperature and then added diisopropyl ether (75 ml). The reaction mass was washed with aqueous acetic acid and further washed with hot water. The solution was concentrated by distillation under reduced pressure at below 55° C. to obtain a residue. The residue was cooled to room temperature and hexane (100 ml) was added to the residue, stirred for 30 minutes at room temperature. The reaction mass further cooled to −10° C. and stirred for 3 hours at same temperature, filtered. The solid obtained was washed with chilled hexane and dried at 40 to 45° C. under reduced pressure to obtain 68 gm of (1′R,2′S,5′R)-menthyl-5(R,S)-acetoxy-[1,3]-oxathiolane-2R-carboxylate.

### Example 3

### Preparation of (2R,5S)-5-(4-amino-2-oxo-2H-pyrimidin-1-yl)-[1,3]-oxathiolane-2-carboxylic acid, 2S-isopropyl-5R-methyl-1R-cyclohexyl ester.

N-propionyl cytosine (125 gm) was suspended in toluene (400 ml) and then added hexamethyl disilazane (135 gm) and methane sulfonic acid (1 gm) at room temperature. The contents were heated to reflux and maintained for 4 hours at reflux. The reaction mass was distilled under atmospheric pressure at 110° C. and then added toluene (250 ml). The contents were cooled to 35° C. and then added methylene chloride (700 ml). The solution was cooled to 15° C. and (1′R,2′S,5′R)-menthyl-5(R,S)-acetoxy-[1,3]-oxathiolane-2R-carboxylate (260 gm) was added to the solution at 15° C. Trityl perchlorate (270 gm) was added at room temperature and heated to 53 to 57° C. The reaction mass was cooled to room temperature and then added methylene chloride (600 ml). Aqueous sodium bicarbonate solution (3300 ml) was added and the layers were separated. The aqueous layer was extracted with methylene chloride at room temperature. Combined the organic layers was washed with aqueous sodium bicarbonate solution. The resulting organic layers were concentrated by distillation under atmospheric pressure at below 57° C. To the residue was added ethanol (100 ml) and stirred for 15 minutes at below 57° C. to obtain slurry. The resulting slurry was concentrated by distillation under reduced pressure at below 57° C. and then maintained for 30 minutes at below 57° C. to obtain a solid residue containing (1′R,2′S,5′R)-menthyl-5S-(N-4″-propionylcytosin-1″-yl)-[1,3]-oxathiolane-2R-carboxylate.

Ethanol (800 ml) was added to the above obtained solid residue containing (1′R,2′S,5′R)-menthyl-5S-(N-4″-propionylcytosin-1″-yl)-[1,3]-oxathiolane-2R-carboxylate at 57° C. The reaction mass was cooled to room temperature and then added methane sulfonic acid (150 gm). Diisopropyl ether (1300 ml) was added and stirred for 50 minutes at room temperature. The reaction mass was cooled to 15° C. and stirred for 40 minutes at 15° C. The separated solid was filtered and washed with a mixture of ethanol and diisopropyl ether to obtain solid. To a solution of ethyl acetate (400 ml) in hexane (100 ml) was added above solid at room temperature. A mixture of triethylamine (40 gm) and hexane (80 ml) was added slowly to the reaction mass. Water (600 ml) was added and stirred for 1 hour at room temperature, filtered. The solid obtained was washed with a mixture of ethyl acetate and hexane and dried at 45 to 50° C. under reduced pressure to obtain 130 gm of (2R,5S)-5-(4-amino-2-oxo-2H-pyrimidin-1-yl)-[1,3]-oxathiolane-2-carboxylic acid, 2S-isopropyl-5R-methyl-1R-cyclohexyl ester.

### Example 4

### Preparation of 4-amino-1-[(2R,5S)-2-(hydroxymethyl)-[1,3]-oxathiolan-5-yl]-(1H)-pyrimidin-2-one Monosuccinate Monohydrate (Lamivudine Succinate Monohydrate)

Dipotassium hydrogen phosphate (116 gm) was dissolved in water (160 ml) and then added ethanol (800 ml) at room temperature. The reaction mass was cooled to 15° C. (2R,5S)-5-(4-amino-2-oxo-2H-pyrimidin-1-yl)-[1,3]-oxathiolane-2-carboxylic acid, 2S-isopropyl-5R-methyl-1R-cyclohexyl ester (130 gm) in methanol (60 ml) was added to the reaction mass at 15° C. A solution of sodium hydroxide (30 mg) in water (170 ml) and then added sodium borohydride (29 gm) under stirring at 12° C. The solution was added to the above reaction mass and maintained for 1 hour at 12° C. The reaction mass was stirred for 30 minutes at room temperature and separated aqueous layer was extracted with ethanol. Combined the organic layers and filtered through hiflo, washed with ethanol. The pH of the filtrate was adjusted to 5.9 to 6.1 with diluted aqueous hydrochloric acid (1:1, 20 ml) and raised the pH immediately to 7.5 to 7.8 with aqueous sodium hydroxide (10% w/v, 25 ml) at 17° C. The reaction mass was concentrate by distillation under atmospheric pressure at below 84° C. to obtain an oily residue. To the residue was added water (450 ml) at 60° C. and cooled to room temperature, the solution was extracted with toluene. To the solution was added carbon enoantiocromos (5 gm) and stirred for 15 minutes, filtered the solution through hyflo and washed with water. To the filtrate was added succinic acid (40 gm) and the contents were stirred for 8 hours at room temperature. The contents were cooled to 5° C. and stirred for 1 hour at same temperature. The separated solid was filtered, washed with chilled water, and then finally the solid was washed with chilled acetone. The solid was dried at 35° C. under reduced pressure for 3 hours to obtain 100 gm of 4-amino-1-[(2R,5S)-2-(hydroxymethyl)-[1,3]-oxathiolan-5-yl]-(1H)-pyrimidin-2-one monosuccinate monohydrate.

### Example 5

### Preparation of 4-amino-1-[(2R,5S)-2-(hydroxymethyl)-[1,3]-oxathiolan-5-yl]-(1H)-pyrimidin-2-one (Lamivudine)

Isopropyl alcohol (900 ml) was dissolved in water (30 ml) and then added 4-amino-1-[(2R,5 S)-2-(hydroxymethyl)-[1,3]-oxathiolan-5-yl]-(1H)-pyrimidin-2-one monosuccinate monohydrate (130 gm) at room temperature. The contents were stirred for 30 minutes at room temperature and then added a mixture of isopropyl alcohol (100 ml) and triethylamine (70 gm). The contents were stirred for 4 hours at room temperature and cooled to 10° C., maintained the contents for 1 hour at the same temperature. The separated solid was filtered, washed with chilled aqueous isopropyl alcohol and dried at 35 to 40° C. under reduced pressure for 6 hours to obtain 60 gm of 4-amino-1-[(2R,5S)-2-(hydroxymethyl)-[1,3]-oxathiolan-5-yl]-(1H)-pyrimidin-2-one (Lamivudine).

### Example 6

### Preparation of N-propionyl-5-fluorocytosine

5-Fluorocytosine (200 gm) was added toluene (800 ml) at room temperature and the contents were heated. The reaction mass was distilled under atmospheric pressure at 110° C. and the contents were cooled to 85° C. Pyridine (180 gm) and dimethylaminopyridine (2 gm) was added to the reaction mass at 85° C. Propionic anhydride (260 gm) in toluene (500 ml) was added under stirring and cooled to room temperature, filtered. The solid obtained was washed with toluene and further washed with water to obtain wet solid. To the wet solid was suspended in water (5000 ml) and triethylamine (10 gm) was added to the suspension at room temperature. The reaction mass was stirred for 3 hours at room temperature, the separated solid was filtered and washed with water. The solid was dried at 50 to 55° C. under reduced pressure for 6 hours to obtain 290 gm of N-propionyl-5-fluorocytosine.

### Example 7

### Preparation of (2R,5S)-5-(4-amino-5-fluoro-2-oxo-2H-pyrimidin-1-yl)-[1,3]-oxathiolane-2-carboxylic acid, 2S-isopropyl-5R-methyl-1R-cyclohexyl Ester.

N-propionyl-5-fluorocytosine (60 gm) was suspended in toluene (200 ml) and then added hexamethyl disilazane (70 gm) and methane sulfonic acid (1 gm) at room temperature. The contents were heated to reflux and maintained for 6 hours at reflux. The reaction was distilled at atmospheric pressure at 110° C. and then added toluene (120 ml). The reaction mass was cooled to 30° C. and then added methylene chloride (300 ml). The solution was cooled to 10° C. and then added (1′R,2′S,5′R)-menthyl-5(R,S)-acetoxy-[1,3]-oxathiolane-2R-carboxylate (130 gm). Trityl perchlorate (140 gm) was added at room temperature. The contents were heated to 50 to 55° C. and cooled to room temperature, and then added methylene chloride (300 ml). Aqueous sodium bicarbonate solution (1500 ml) was added and separated aqueous layer was extracted with methylene chloride at room temperature. Combined the organic layers was washed with aqueous sodium bicarbonate solution and the resulting organic layers were concentrated by distillation under atmospheric pressure at below 57° C. To the residue was added ethanol (50 ml) and stirred for 15 minutes at below 57° C. to obtain slurry. The resulting slurry was concentrated by distillation under reduced pressure at below 57° C. and then maintained for 30 minutes at below 57° C. to obtain a solid residue.

Methanol (400 ml) was added to the above obtained solid residue at 57° C. The reaction mass was cooled to room temperature and then added methane sulfonic acid (70 gm). Diisopropyl ether (600 ml) was added and stirred for 50 minutes at room temperature. The reaction mass was cooled to 15° C. and stirred for 1 hour at same temperature. The separated solid was filtered and washed with a mixture of ethanol and diisopropyl ether to obtain solid. To a solution of ethyl acetate (200 ml) in hexane (100 ml) was added above solid at room temperature. A mixture of triethylamine (20 gm) and cyclohexane (40 ml) was added slowly to the reaction mass. Water (300 ml) was added and stirred for 1 hour at room temperature, filtered. The solid obtained was washed with a mixture of ethyl acetate and hexane and dried at 45 to 50° C. under reduced pressure to obtain 60 gm of (2R,5S)-5-(4-amino-5-fluoro-2-oxo-2H-pyrimidin-1-yl)-[1,3]-oxathiolane-2-carboxylic acid, 2S-isopropyl-5R-methyl-1R-cyclohexyl ester.

### Example 8

### Preparation of 4-amino-5-fluoro-1-[(2R,5S)-2-(hydroxymethyl)-[1,3]-oxathiolan-5-yl]-(1H)-pyrimidin-2-one Monosuccinate Monohydrate (Emtricitabine Succinate Monohydrate)

Ethanol (350 ml) was added to a solution of dipotassium hydrogen phosphate (52 gm) in water (70 ml) at room temperature. The reaction mass was cooled to 12° C. and (2R,5S)-5-(4-amino-5-fluoro-2-oxo-2H-pyrimidin-1-yl)-[1,3]-oxathiolane-2-carboxylic acid, 2S-isopropyl-5R-methyl-1R-cyclohexyl ester (59 gm) in methanol (30 ml) was added to the reaction mass. A solution of sodium hydroxide (10 mg) in water (70 ml) and then added sodium borohydride (12 gm) under stirring at 12° C. The solution was added to the above reaction mass and maintained for 2 hour at 12° C. The reaction mass was stirred for 1 hour at room temperature and separated aqueous layer was extracted with ethanol. Combined the organic layers and filtered through hiflo, washed with ethanol. The pH of the filtrate was adjusted to 5.9 to 6.2 with diluted aqueous hydrochloric acid (1:1, 10 ml) and raised the pH immediately to 7.6 to 7.8 with aqueous sodium hydroxide (10% w/v, 15 ml) at 17° C. The reaction mass was distilled under atmospheric pressure at below 84° C. To the residue was added water (200 ml) at 60° C. and cooled to room temperature, the solution was extracted with toluene. To the solution was added carbon enoantiocromos (2 gm) and stirred for 15 minutes, filtered the solution through hyflo and washed with water. To the filtrate was added succinic acid (20 gm) and the contents were stirred for 10 hours at room temperature. The contents were cooled to 0° C. and stirred for 1 hour at same temperature. The separated solid was filtered and washed with chilled acetone. The solid was dried at 35° C. under reduced pressure for 4 hours to obtain 40 gm of 4-amino-5-fluoro-1-[(2R,5S)-2-(hydroxymethyl)-[1,3]-oxathiolan-5-yl]-(1H)-pyrimidin-2-one monosuccinate monohydrate.

### Example 9

### Preparation of 4-amino-5-fluoro-1-[(2R,5S)-2-(hydroxymethyl)-[1,3]-oxathiolan-5-yl]-(1H)-pyrimidin-2-one (Emtricitabine)

4-amino-5-fluoro-1-[(2R,5S)-2-(hydroxymethyl)-[1,3]-oxathiolan-5-yl]-(1H)-pyrimidin-2-one monosuccinate monohydrate (40 gm) was added to a solution of isopropyl alcohol (260 ml) in water (20 ml) at room temperature. A mixture of isopropyl alcohol (30 ml) and triethylamine (30 gm) was added and stirred for 5 hours at room temperature. The contents were cooled to 12° C. and maintained 2 hour at 12° C., filtered and washed with chilled aqueous isopropyl alcohol. The solid was dried at 40 to 45° C. under reduced pressure for 8 hours to obtain 19 gm of 4-amino-5-fluoro-1-[2R,5S)-2-(hydroxymethyl)-[1,3]-oxathiolan-5-yl]-(1H)-pyrimidin-2-one (Emtricitabine).

1. A process for the preparation of nucleosides, which comprises:
a) reacting the compound of formula I: 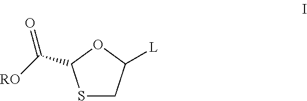
wherein the R is independently hydrogen and optionally substituted alkyl groups and L is leaving group with a compound of formula II: 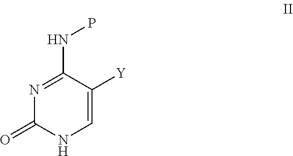
wherein the P is protecting group and Y is hydrogen or fluorine to obtain a compound of formula III: 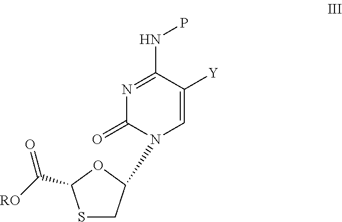
wherein the R, P and Y are defined above,
b) treating the compound of formula III with an acid to obtain a compound of formula IV; 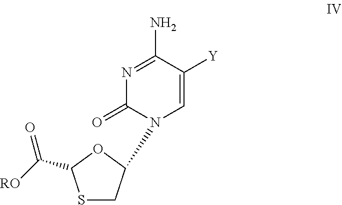
wherein the R and Y are defined above,
c) reducing the compound of formula IV with a reducing agent to obtain a compound of formula V, 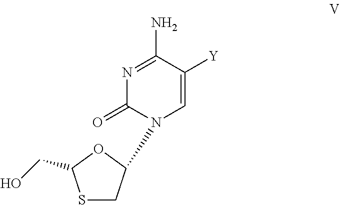
wherein the Y is defined above,
d) optionally, converting the compound of formula V to a salts thereof.

2. The process according to claim 1, wherein R is an optionally substituted alkyl group.

3. The process according to claim 2, wherein the substituted alkyl group is a chiral auxiliary.

4. The process according to claim 3, wherein the chiral auxiliary is selected from (d)-menthyl, (l)-menthyl, (d)-8-phenylmenthyl, (l)-8-phenylmenthyl, (+)-norephedrine and (−)-norephedrine.

5. The process according to claim 4, wherein the chiral auxiliary is (d)-menthyl or (l)-menthyl.

6. The process according to claim 1, wherein the leaving group is selected from acetoxy, ethoxy carbonyl, iodine, bromine, chlorine or fluorine.

7. The process according to claim 6, wherein the leaving group is acetoxy.

8. The process according to claim 1, wherein protecting group is selected from propionyl, butanoyl, pentanoyl, hexanoyl, tosyl, mesyl or benzoyl.

9. The process according to claim 8, wherein protecting group is propionyl.

10. The process according to claim 1, wherein the acid used in step (b) is selected from methane sulfonic acid, ethane sulfonic acid, p-toluene sulfonic acid, acetic acid, formic acid, hydrochloric acid, sulfuric acid or phosphoric acid.

11. The process according to claim 10, wherein the acid is methane sulfonic acid.

12. The process according to claim 1, wherein the reaction in step (b) is carried out in a solvent or mixture thereof.

13. The process according to claim 12, wherein the solvent is selected from ether solvents are diisopropyl ether, di-tert-butyl ether, diethyl ether, 1,4-dioxane, ethyl tert-butyl ether, methyl tert-butyl ether and tetrahydrofuran.

14. The process according to claim 13, wherein the ether solvent is diisopropyl ether.

15. The process according to claim 1, wherein the reducing agent used in step (c) is selected from sodium borohydride, lithium aluminium hydride, sodium amalgam, oxalic acid, formic acid or diisobutylalumiminum hydride.

16. The process according to claim 15, wherein the reducing agent is sodium borohydride.

17. The process according to claim 1, wherein the acid addition salts prepared in step (d) are hydrochloric acid, sulfuric acid, methane sulfonic acid, succinic acid, salicylic acid, malic acid and p-toluene sulfonic acid.

18. The process according to claim 17, wherein the acid addition salt is succinic acid.
